(19) United States
(12) Patent Application Publication
(10) Pub. No.: US 2015/0366763 A1
(43) Pub. Date: Dec. 24, 2015

(54) Eyeliner Decal Composition and Method of Use

(71) Applicant: Alexandra Barilari, Gardena, CA (US)

(72) Inventor: Alexandra Barilari, Gardena, CA (US)

(21) Appl. No.: 14/838,324

(22) Filed: Aug. 27, 2015

(51) Int. Cl. A61K 8/02; A61K 8/34; A61K 8/81; A61K 8/41; A61K 8/97; A45D 40/30; A61K 8/19; A61K 8/60; A61K 8/37; A61K 8/86; A61K 8/73; A61K 8/92; A61Q 1/10; A61K 8/891

(52) U.S. Cl. CPC A61K 8/0204; A61Q 1/10; A61K 8/345; A61K 8/8194; A61K 8/41; A61K 8/97; A61K 8/891; A61K 8/19; A61K 8/60; A61K 8/8152; A61K 8/37; A61K 8/34; A61K 8/86; A61K 8/731; A61K 8/922; A61K 8/817; A61K 8/8182; A61K 8/8147; A45D 40/30; A61K 2800/87; A61K 2800/43

(57) ABSTRACT

The present invention relates to compositions including humectant, colorant, matting agent, pH modifier, film forming agent, rheology modifier, and adhesive, as well as methods of producing such compositions and using such compositions for cosmetics especially adapted for use as eyeliners.

### FIELD OF THE INVENTION

The present invention generally relates to cosmetic compositions and method of use. More particularly, the present invention is directed to a temporary eyeliner composition that can be adhered to eyelids and subsequently removed therefrom.

### BACKGROUND OF THE INVENTION

Eyeliner is a popular cosmetic used to define the eyes. It is generally applied around the contours of the eyes along the rim. Eyeliners of varying degrees of thickness and style create a variety of aesthetic effects. For example, winged eyeliners draw attention to the eye and can enhance or even change the shape of the eye. Additionally, eyeliners can make the eyelashes look more full, lush, and long.

Eyeliners are typically applied with a pencil, brush, or a tipped sponge, but the process of applying eyeliners accurately and evenly with these instruments is difficult. Particularly, using these instruments requires a user to have a steady hand so that the user can draw smooth lines closely along the rim of the eyes. Additionally, the user must apply the eyeliner in a precise manner to make sure that the eyeliner on one eye matches the eyeliner on the other eye. Applying eyeliners, therefore, can be a laborious and a time consuming process that can cause great annoyance. In this regard, there is a need in the prior art for eyeliner that permits instant, precise application in order to increase convenience to the user while enhancing the appearance of the user's eyes.

Various types of cosmetics that can be temporarily worn on a person's face, and that claim compositions for the same exist in the prior art. These include eyeliners and cosmetic compositions that have been patented and published in patent application publications. The foregoing is a list of devices deemed most relevant to the present disclosure, which are herein described for the purposes of highlighting and differentiating the unique aspects of the present invention, and further highlighting the drawbacks existing in the prior art.

Specifically, Tanielian (U.S. Patent Application Publication No. 2014/0011003), Dobler (U.S. Patent Application Publication 2013/0337225), Hurwitz (U.S. Patent Application Publication No. 2010/0239619), and Dunlap-Harris (U.S. Pat. No. 6,161,554) disclose temporary facial decals that can be applied to the face. These decals are generally printed on a sheet of paper and protected via a transparent backing layer, which can be removed when the decals are ready for use. Though the foregoing prior art references disclose decals that can be transferred to a person's face, these decals are limited in that they are not configured to be worn around the rim of the eyes or along the edges of the eyelids.

Similarly, Maxey (U.S. Patent Application Publication No. 2010/0242539) and Weld (U.S. Pat. No. 3,266,500) disclose a decorative eyelid covering and a combination eyeliner and artificial eyelash structure, respectively. Each of these devices comprises an adhesive backing that can be directly attached to a user's eyelid, similar to a sticker. In this regard, these devices are not transferred to the user's eyelids when applied, as disclosed in the present invention. Therefore, Maxey and Weld do not disclose an eyeliner as described in the present invention.

Some prior art references disclose cosmetic compositions. Particularly, Dempsey (U.S. Patent Application Publication No. 2012/0114585), Scancarella (U.S. Patent Application Publication No. 2003/0017123), and Manelski (U.S. Pat. No. 6,458,390) disclose makeup compositions that are long-lasting. These compositions, however, are semi-solid or viscous liquid in form. Specifically, Dempsey discloses a composition specifically for mascara; and Manelski discloses a single-phase gel based makeup composition. Thus, these compositions cannot be used for producing solid decals that can be printed and then adhered to a user's face.

The eyeliners and compositions thereof disclosed in the prior art have several known drawbacks, however. These eyeliners and compositions are limited in that they are not configured to be used as a decal, and that can be worn on the eyelids as eyeliners. The present invention overcomes these limitations by disclosing an eyeliner decal composition comprising a solvent/humectant agent, pigment, matting agent, pH modifier, film forming agent, rheology modifier, and adhesive.

### SUMMARY OF THE INVENTION

In view of the disadvantages inherent in the known types of devices for eyeliners now present in the prior art, the present invention provides an improved eyeliner composition and method of use.

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as to prelude to the more detailed description that is disclosed later.

The present invention relates to compositions including humectant, colorant, matting agent, pH modifier, film forming agent, rheology modifier, and adhesive, as well as methods of producing such compositions and using such compositions for cosmetics especially adapted for use as eyeliners. In a preferred embodiment, the present invention comprises 38.87% glycerin, 1.28% Carbon Black, 1.09% Synafil W1234, 1.82% triethanolamine, 56.75% Centex LATZ latex, and 0.18% BYK 333. All of the measurements are provided in percentage by weight relative to the total weight of the composition.

Another aspect of the present invention provides an eyeliner decal containing one or more compositions according to the description herein. More specifically, the decal can be applied to the eyelids via a transfer paper and worn similarly to conventional eyeliners. Because the decals comprise pre-printed designs, the present invention provides a means for applying different eyeliner designs in a precise and convenient manner.

It is therefore an objective of the present invention to provide an eyeliner decal that can be worn easily and that enhances appearance of the eyes.

It is another objective of the present invention to provide an eyeliner decal composition that can be worn for an extended period of time without smudging or detaching from the user's eyelids.

It is still another objective of the present invention to provide an eyeliner decal composition that can be worn safely on the rim of the eyelids.

It is still another objective of the present invention to provide an eyeliner decal composition that is flexible and that can conform to the contours of an eyelid.

It is still another objective of the present invention to provide an eyeliner decal composition that can be printed in various designs and shapes to increase versatility in appearance.

It is still another objective of the present invention to provide an eyeliner decal composition that comprises variety of colors and texture.

It is still another objective of the present invention to provide an eyeliner decal composition that can be sparkly, glittery, shiny, and luminous in appearance.

It is still another objective of the present invention to provide an eyeliner decal composition that can be removed without using a make up remover.

A final objective of the present invention to provide an eyeliner decal composition that may be readily fabricated from materials that permit relative economy and commensurate with durability.

In the light of the foregoing, these and other objects are accomplished in accordance of the principles of the present invention, wherein the novelty of the present invention will become apparent from the following detailed description and appended claims.

### BRIEF DESCRIPTION OF THE DRAWINGS

The above and other objects and advantages of the present invention will be apparent upon consideration of the following detailed description, taken in conjunction with the accompanying exemplary drawings, in which like reference characters refer to like parts throughout, and in which:

FIG. 1 shows a protective backing being removed from a printed eyeliner decal on a transfer paper.

FIG. 2 shows a printed eyeliner decal being attached to a user's eyelid.

FIG. 3 shows a transfer paper being removed from a user's eyelid.

FIGS. 4 through 17 show various embodiments of the eyeliner decal of the present invention.

### DETAILED DESCRIPTION OF THE INVENTION

The present invention is directed towards eyeliner decal compositions and method of use. For purposes of clarity, and not by way of limitation, illustrative views of the present eyeliner decal composition are described with references made to the above-identified figures. Various modifications obvious to one skilled in the art are deemed to be within the spirit and scope of the present invention.

The word “exemplary” is used herein to mean serving as an example, instance, or illustration. Any aspect or design described herein as “exemplary” is not necessarily to be construed as preferred or advantageous over other aspects or designs. Rather, use of the word exemplary is intended to disclose concepts in a concrete fashion. As used in this application, the term “or” is intended to mean an inclusive “or” rather than an exclusive “or.”

Additionally, the articles “a” and “an” as used in this application and the appended claims should generally be construed to mean “one or more” or “at least one,” thus including individual components as well as mixtures/combinations, unless specified otherwise or clear from context to be directed to a singular form. Thus, the cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to eyeliners.

“Water-resistant” as used herein refers to the ability to resist the penetration of water to some degree but not entirely. Water-resistant properties may be evaluated by any method known in the art for evaluating such properties. For example, an eyeliner composition may be applied to a substrate or skin, and then dried until set. Once the eyeliner composition is dried, the substrate or skin may be submerged in water for a certain amount of time or splashed with water repeatedly a certain number of times. After the expiration of the pre-determined amount of time, the eyeliner on the substrate or skin may be evaluated for any visible smearing or smudging. A composition may be water-resistant if a majority of the product is left on the substrate or skin.

It is contemplated, however, that the composition herein may be also formulated as washable. The term “washable,” as used herein, refers to the compositions that generally can be removed with water and/or soap. These formulations are typically emulsions (e.g., of waxes in water). Water-resistant compositions, on the other hand, typically require use of oils for removal. Such compositions generally come in the form of dispersions of waxes in organic solvents.

“Transfer resistance” as used herein refers to the quality exhibited by compositions that are not readily removed by contact with another materials, such as, for example, an item of clothing or the skin. Transfer resistance may be evaluated by any method known in the art for evaluating such. For instance, transfer resistance of a composition may be evaluated by applying the composition to the skin and then rubbing a material with the skin, for example, a sheet of paper, after expiration of a certain amount of following application, such as after the composition is set.

Similarly, transfer resistance of a composition may be evaluated by the amount of product transferred from a wearer to any other substrate, such as transfer from the lips, skin, or eyelashes of an individual to clothing after the expiration of a certain amount of time following application. The amount of composition transferred to the substrate may then be evaluated and compared. It is contemplated that a composition may be transfer resistant if a majority of the product is left on the skin. Further, the amount of transferred may be compared with that transferred by other compositions, such as commercially available compositions or alternate embodiments.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances. Work of adhesion or tackiness can be quantified using acceptable techniques and methods generally used to measure adhesion.

### Solvents/Humectant Agents

According to the present invention, compositions comprising at least one solvent, wherein said solvent is a humectant agent such as glycerin are provided. Glycerin provides a medium to adequately mix the pigments and matting agent, along with providing the soft feel by preventing the film from completely drying out. Preferred glycerin include those that are commercially available from the company Duda Energy LLC. Without limitation, other suitable solvents/humectant agents include: ethyl lactate, ethanol, sorbitol, alcohols, water, ethers, sugar alcohols, urea, glycols, polyols, collagen, acetates, aloe vera (aloe barbadensis leaf juice), coconut oil (cocos nucifera), and honey.

According to preferred embodiments, the glycerin is present in an amount sufficient to reduce dryness of the composition. It is to be understood that the amount of glycerin needed to reduce dryness of a composition should depend upon the amount of pigments, e.g., colorant, matting agent, e.g., polypropylene, and pH modifier present in the composition. For example, the more pigment and matting agent which are present, the more glycerin should be added to reduce dryness of the composition.

In a particularly preferred embodiment, sufficient glycerin is present in the composition to substantially eliminate dryness of the composition and to create a soft feel when it is set and forms a film-like consistency. Generally speaking, preferred ranges of glycerin in the composition are from about 20.0% to 60.0% by weight of the total weight of the composition, including all ranges and subranges therebetween, and more preferably about 38.87% of the total weight of the composition.

### Colorant

According to the present invention, the compositions may comprise at least one coloring agent. Carbon black provides the black color that is desired. Preferred carbon black include those that are commercially available from the company MakingCosmetics Inc. Non-limiting examples of other suitable colorants include: iron oxides, charcoals, micas, pearlescent pigments, metallic pigments, talc, clay, kaolin, zinc oxide, nylon powder, ultra marine, titanium dioxide, titanated mica, calcium carbonate, tar pigments, organic pigments, FD&C dyes, D&C dyes, and/or a combinations thereof.

Although a black colorant is generally used, colorants having characteristics of being red, orange, yellow, green, blue, purple, pink, white, brown, and combinations of thereof may also be used, depending upon embodiment. The pigments may be white, colored, inorganic, organic, polymeric, nonpolymeric, coated and uncoated pigments.

For example, coated pigments such as nacreous pigments comprising thin flakes with one or more layers of metal oxide including titanium dioxide, bismuth oxychloride, and/or iron oxide may be used. Light diffraction and interferences occur in these layers produce a pearlescent and luster effect. Pearlescent effects can also be achieved by choosing white nacreous pigments such as mica coated with titanium or with bismuth oxychloride. Alternatively, colored nacreous pigments such as titanium mica with iron oxides, titanium mica with ferric blue or chromium oxide, titanium mica with an organic pigment, and nacreous pigments based on bismuth oxychloride may be used.

The pigments, including nacreous pigments, may be present in the composition in a range from about 0.10% to 10.0% by weight of the total weight of the composition, including all ranges and subranges therebetween, and more preferably 1.28% of the total weight of the composition.

### Matting Agent

Polypropylene provides adequate matting while remaining compatible within the coating. It is to be understood that the amount of polypropylene needed to mattify a composition should depend upon the desired affect or the appearance. For example, the more polypropylene which are present, the more matte the appearance of the composition. Other matting agents include: aluminum oxides, silicas, propylenes, waxes, e.g., microcrystalline wax, acrylics, sulfates, polyester, silicates, carbonates, zinc oxides, and titanium oxides.

Preferred polypropylenes include those that are commercially available from the company Eckart America under the name Synafil W1234, which is a cosmetic filler based on synthetic fluorphlogopite and polypropylene wax. A polypropylene that is particularly preferred is in the form of free flowing white powder comprising micronized polypropylene wax. When present, care should be taken that the matting agent is used at a level of no more than 5% (w/w) to avoid undesirable or unnatural opaque appearance. Generally speaking, preferred ranges of polypropylene in the composition are from about 0.1% to 5.0% by weight of the total weight of the composition, including all ranges and subranges therebetween, and more preferably 1.09% of the total weight of the composition.

### pH Modifier/Emulsifier

According to preferred embodiments of the present invention, the composition further comprises at least one pH modifier/emulsifier. Suitable pH modifier/emulsifier for use in accordance with the present invention include, but are not limited to, triethanolamine, which increases the pH of the composition to improve compatibility of the various additives to the latex film-forming layer. Preferred triethanolamine include those that are commercially available from the company Spectrum Chemical. Non-limiting examples of other suitable pH modifier/emulsifier include trolamine, sodium hydroxide, acetic acid, lactic acid, sodium laureth sulfate, polyacrylic acid, sodium lauryl sulfate, and sodium acetate.

The amount of pH modifier/emulsifier to be added may be calculated in order to achieve a suitable pH, which results in an improved compatibility of the various additives to the latex film-forming layer. Care should be taken that the pH is not too high, such that it would cause the composition to be too dry and therefore cause the eyeliner to appear lackluster. As such the pH may be optimized according to the parameters of cosmetically acceptable values.

According to preferred embodiments, the pH modifier(s) is/are present in the composition in an amount ranging from 0.1% to 5.0% by weight relative to the total weight of the composition, including all ranges and subranges therebetween, and more preferably 1.82% of the total weight of the composition.

### Film Forming Agent

According to preferred embodiments of the present invention, the composition further comprises at least one film forming agent, such as latex, which forms a flexible, sturdy film. Preferred latex includes those that are commercially available from the company Centrotrade Minerals & Metals, Inc. under the name Centex LATZ, which is available in the form of a dilute solution. Other cosmetic film forming agents include: sucrose acetate isobutyrate, dermacryl C, polyethylene glycol (PEG) 8000, ethyl cellulose, cellulosic polymers, acrylics/acrylates, carbomers, polyesters, nylons, synthetic rubbers, silicones, polyurethane/polyuria dispersions, polyvinyl alcohol, polyvinylpyrrolidone, acrylamides, other thermoplastic polymers, and other compounds or group of compounds which, when added to latex, favors film-forming.

The film forming agents preferably prevent or helps prevent latex coagulation while in contact with the skin, not substantially affecting the polymerization of the compounds contained in the latex. Polymucosaccharides such as glucosaminoglycans also provide such property, particularly one or more from hyaluronic acid, hyaluronates of metals such as sodium, heparin, chondroitin-sulphate, acaran sulphate, keratin sulphate, or dermatan sulphate. Other considered as film-forming agents are natural polysaccharides such as starch, amylose and amylopectin, cellulose and its derivatives, chitosan, alginate, agar, agarose and their mixtures among themselves or with others.

According to preferred embodiments, the film forming agent(s) is/are present in the composition in an amount ranging from 20% to 60% by weight relative to the total weight of the composition, including all ranges and subranges therebetween, and more preferably 56.75% of the total weight of the composition.

### Rheology Modifier

According to preferred embodiments of the present invention, the composition further comprises at least one rheology modifier. For instance, polyether-modified polydimethylsiloxane modified the surface tension of the system to provide an even surface upon curing. Preferred polyether-modified polydimethylsiloxane include those that are commercially available from the company Altana under the name BYK 333, which is a silicone-containing surface additive for solvent-free, solvent-born, and aqueous coating systems, printing inks, and adhesive systems as well as ambient curing plastic systems. Additionally, BYK 333 has strong reduction of surface tension.

Non-limiting examples of rheology modifiers include water-soluble polymers selected from the group consisting of: anionic/cationic/neutral surfactants; silica/silicates; silicones; and fluoropolymers. Additionally, other rheology modifiers such as: cetyl alcohols; polyvinyl alcohols; hydroxyalkyl starches; hydroxyl ethyl celluloses; hydroxypropyl celluloses; carboxylated latexes; hydroxylated latexes; alginates; polyacrylates; natural gums; modified gums (e.g., guar gum); polyethylene oxide; polypropylene oxide;) proteins (e.g., gelatin); glycoprotein; polysaccharides (e.g., starch, extracts from plants, such as okra and sassafras); capsular polysaccharides; polyacrylamides; polyvinylpyrrolidine; cellulose derivatives; mucin powder; nucleic acids; and combinations thereof may be used.

According to preferred embodiments, the rheology modifier(s) is/are present in the composition in an amount ranging from 0.1% to 1.0% by weight relative to the total weight of the composition, including all ranges and subranges therebetween, and more preferably 0.18% of the total weight of the composition.

### Additional Ingredients

The composition of the present invention can also comprise any additive usually used in the field under consideration. In particular, other solvents, film forming agents, oils, thickeners, adhesives, preserving agents, cosmetic and dermatological active agents such as emollients, moisturizers, vitamins, sunscreens, and mixtures thereof can be added. More specifically, it is contemplated that the foregoing additional ingredients are those ordinarily used in the art, and are suitable for cosmetics use. For example, preserving agents having antimicrobial properties such as benzyl alcohol, paraben, and other suitable preservatives may be used to prolong the life of the composition prior to usage thereof.

### Adhesive

Optionally, the present composition further comprises at least one adhesive. Vinyl acrylic polymer provided the proper level of adhesion that also could be removed in the presence of water, makeup remover, or other skin safe cleaners including organic solvents such as ethanol. Preferred vinyl acrylic polymer includes those that are commercially available from the company Akron Coating and Adhesives Inc. under the name ACA-20-113. Other adhesives include: polyvinyl alcohol, polyvinyl acetate, acrylics/acrylates, nitrocellulose, ethylene vinyl acetate, starch, and epoxies.

If present, the amount of adhesive to be added may be calculated in order to achieve a suitable adhesion so as to allow the composition to temporarily attach to the skin. Care should be taken that the amount of adhesive is not too high or too low, such that it would cause the composition to be too sticky or unreliable.

The present invention also comprises eyeliner decals, kits, and/or prepackaged materials suitable for cosmetic use containing one or more compositions according to the description herein.

### EXAMPLE 1

An eyeliner composition was made according to the following formula in Table 1:

TABLE 1

| Trade Name | INCI Name | % (w/w) |
|---|---|---|
| Glycerin | Glycerin | 20-60 |
| Carbon Black (D&C | Carbon Black | 0.1-10 |
| Black No. 2) | | |
| Synafil W1234 | Polypropylene | 0.1-5 |
| Triethanolamine | Triethanolamine | 0.1-5 |
| Centex LATZ Latex | Latex | 20-60 |
| BYK 333 | Polyether-modified | 0.1-1 |
| | polydimethylsiloxane | |
| | TOTAL | 100.0 |

### Process

1. Glycerin, Synafil W1234, Carbon Black (i.e., D&C Black No. 2) were combined in a mixing container and mixed until Synafil W1234 and Carbon Black were well dispersed in glycerin.

2. The mixture was continuously stirred under medium shear and triethanolamine was slowly added to the mixing container.

3. The speed of the mixture was reduced to slight agitation and then Centex LATZ latex was slowly added to the mixing container.

4. After Centex LATZ was incorporated, BYK 333 was slowly added to the mixing container. Care should be taken to prevent Centex LATZ latex and BYK 333 from being added too abruptly or under too high of a shear as this can cause the latex to begin to polymerize in the solution.

5. Once all ingredients were dissolved and mixed well, an initial film was roll coated at 20 wet mils on a slip paper, transfer paper, decal paper, and/or the like.

6. The film was allowed to partially dry in an oven set at 100° C.±5° C. before being removed from the oven and pressed flat to 5 mils or less.

7. Optionally, a layer of adhesive may be evenly applied over the film.

8. The film was then placed back in the oven and was allowed to dry completely before being removed and allowed to cool in ambient room temperature (e.g., 23° C. to 25° C.). Partially drying the film allows the film to be compressed, forming a thinner and more flexible coating without smearing.

9. After the film was allowed to cool, it was cut into a desired decal shape via stamping, die cutting, and/or other suitable methods. Other potential means of applying the coating include spraying the coating onto the slip paper, roll coating, reverse roll coating, vapor deposition, inkjet, silk screen, stencil, gravure coatings.

### EXAMPLE 2

An eyeliner composition were made according to the following formula in Table 2:

TABLE 2

| Trade Name | INCI Name | % (w/w) | % (w/w) |
|---|---|---|---|
| Sucrose Acetate | Sucrose Acetate | 30-50 | 46 |
| Isobutyrate | Isobutyrate | | |
| Dermacryl C | Acrylates Copolymer | 10-20 | 14 |
| Ethyl Lactate | Ethyl Lactate | 20-50 | 31 |
| Ethanol | Ethanol | 1-10 | 9 |
| | TOTAL | 100.0 | 100.0 |

### EXAMPLE 3

An eyeliner composition were made according to the following formula in Table 3:

TABLE 3

| Trade Name | INCI Name | % (w/w) | % (w/w) |
|---|---|---|---|
| Ethanol | Ethanol | 50-95 | 83 |
| PEG 8000 | Polyethylene Glycol | 0.25-5 | 1 |
| Ethyl Cellulose 7 | Ethyl Cellulose | 1-15 | 7 |
| Coconut Oil | Cocos Nucifera | 1-5 | 2 |
| (fractionalized) | (fractionalized) | | |
| Iron Oxide Black | Iron Oxide Black | 0.1-5 | 2 |
| Polyvinyl Alcohol | Polyvinyl Alcohol | 1-15 | 5 |
| | TOTAL | 100.0 | 100.0 |

### EXAMPLE 4

An eyeliner composition were made according to the following formula in Table 4:

TABLE 4

| Trade Name | INCI Name | % (w/w) | % (w/w) |
|---|---|---|---|
| Ethanol | Ethanol | 60-95 | 75 |
| Microcrystalline Wax | Microcrystalline Wax | 1-5 | 4 |
| Ethyl Cellulose 30 | Ethyl Cellulose | 5-20 | 7 |
| Synafil W1234 | Polypropylene | 1-5 | 3 |
| Coconut Oil | Cocos Nucifera | 1-5 | 2 |
| (fractionalized) | (fractionalized) | | |
| Sodium Lauryl Sulfate | Sodium Lauryl Sulfate | 0.1-5 | 0.1 |
| Iron Oxide Black | Iron Oxide Black | 0.1-5 | 1.9 |
| 10% Polyvinyl | 10% Polyvinyl Alcohol | 1-15 | 7 |
| Alcohol in Water | in Water | | |
| | TOTAL | 100.0 | 100.0 |

### EXAMPLE 5

An eyeliner composition were made according to the following formula in Table 5:

TABLE 5

| Trade Name | INCI Name | % (w/w) | % (w/w) |
|---|---|---|---|
| Ethanol | Ethanol | 50-90 | 82 |
| Cetyl Alcohol | Cetyl Alcohol | 1-10 | 4 |
| Ethyl Cellulose 7 | Ethyl Cellulose | 5-15 | 7 |
| Synafil W1234 | Polypropylene | 0.1-5 | 2.6 |
| Sorbitol | Sorbitol | 0.1-10 | 2 |
| Sodium Laureth | Sodium Laureth Sulfate | 0.1-5 | 0.5 |
| Sulfate | | | |
| Carbon Black (D&C | Carbon Black | 0.1-5 | 1.9 |
| Black No. 2) | | | |
| | TOTAL | 100.0 | 100.0 |

### EXAMPLE 6

An eyeliner composition were made according to the following formula in Table 6:

TABLE 6

| Trade Name | INCI Name | % (w/w) | % (w/w) |
|---|---|---|---|
| Centex LATZ Latex | Latex | 10-50 | 32 |
| Glycerin | Glycerin | 20-80 | 40 |
| Triethanolamine | Triethanolamine | 1-15 | 7 |
| Synafil W1234 | Polypropylene | 0.1-5 | 2.6 |
| BYK 333 | Polyether-modified | 0.1-5 | 0.5 |
| | polydimethylsiloxane | | |
| ACA-20-113 | Vinyl Acrylic Polymer | 5-20 | 16 |
| Carbon Black (D&C | Carbon Black | 0.1-5 | 1.9 |
| Black No. 2) | | | |
| | TOTAL | 100.0 | 100.0 |

### EXAMPLE 7

An eyeliner composition were made according to the following formula in Table 7:

TABLE 7

| Trade Name | INCI Name | % (w/w) | % (w/w) |
|---|---|---|---|
| Glycerin | Glycerin | 20-80 | 30 |
| Dermacryl C | Acrylates Copolymer | 10-50 | 25 |
| Polyacrylic Acid | Polyacrylic Acid | 10-50 | 25 |
| Deionized Water | Deionized Water | 10-60 | 18 |
| Iron Oxide Black | Iron Oxide Black | 0.1-5 | 2 |
| | TOTAL | 100.0 | 100.0 |

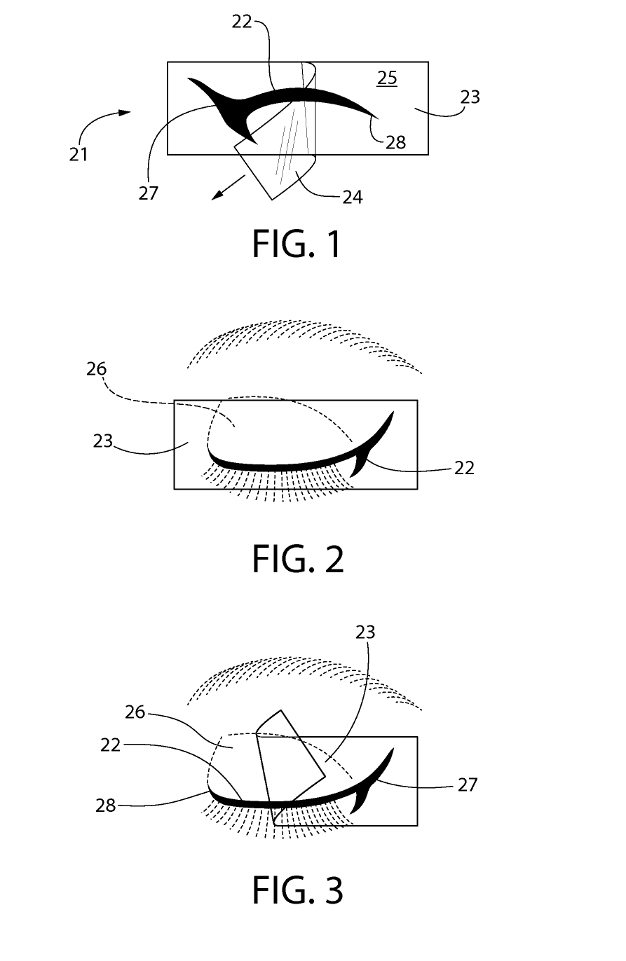

Another aspect of the present invention is a method of use of the eyeliner decal, characterized by comprising the following steps, as illustrated in FIGS. 1 through 3:

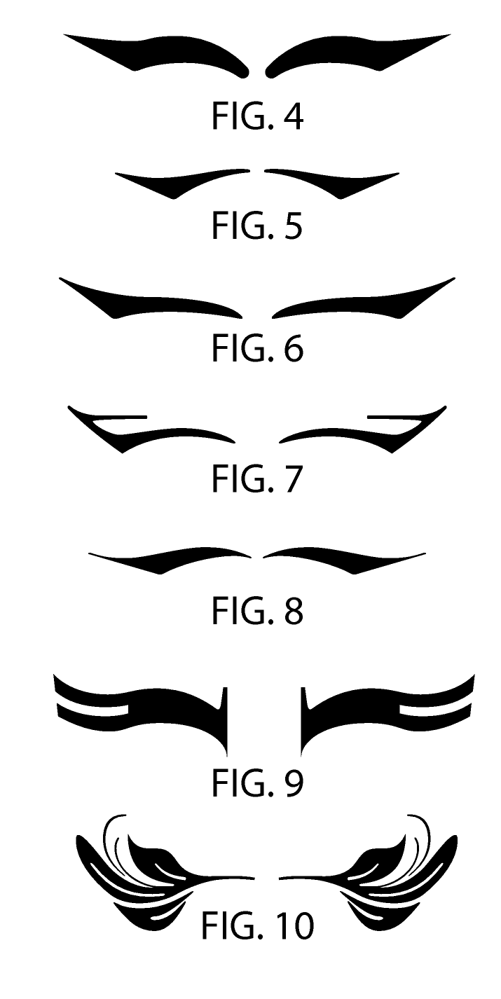

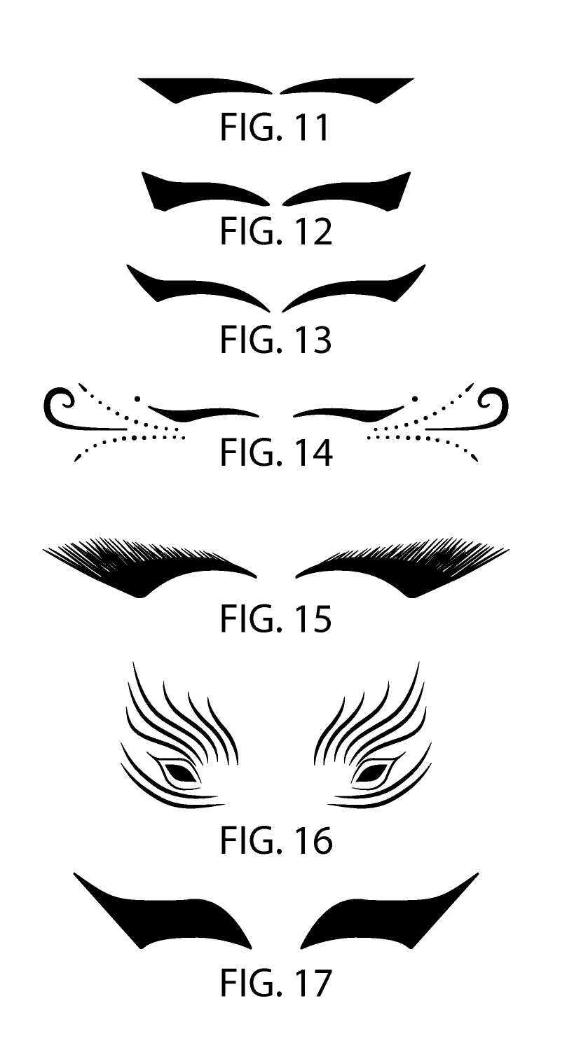

The present invention comprises an eyeliner decal 21 having a protective backing layer 24 removably attached to a slip paper 23 having a decal 22 printed on an application surface 25 thereof. It is preferred that the slip paper 23 is substantially transparent so as to allow a user to see through and determine the positioning of the decal 22 when placing the slip paper 23 over the eyes. The decal 22 comprises a variety of shapes and sizes, as depicted in FIGS. 4 through 17. The decal 22 comprises a first end 28 and a second end 27, wherein the first end 28 is configured to align with the inner corner of the eye and the second end 27 is configured to align with the outer corner of the eye.

1. In use, the removable backing layer 24 is gently removed from the slip paper 23 to prevent tearing the decal 22 prior to use.

2. The slip paper 23 is then positioned over a user's eye 26 such that the decal 22 faces the eye. Preferably, the user should practice placement of the decal 22 before applying the decal 22.

3. The decal 22 should be placed on the eye such that the second end 27 aligns with the outer corner of the eye 26 and that the decal 22 is positioned as close to the rim of the eyelid as possible. The user should take care to prevent touching the decal 22 as the oil from the fingers can prevent the decal 22 from properly attaching to the skin.

4. Once in a desired position, the slip paper 23 should be gently pressed to the eye, taking care that eyelashes or other debris are not stuck under the decal 22. In this regard, it is highly preferred that the skin be freshly cleansed without oils or makeup. Thus, the eyeliner should be applied before any other makeup is applied to the face.

5. After the slip paper 23 is substantially applied to the eye 26, the slip paper 23 is moistened with water until completely wet, preferably from the second end of the decal 22. The water should assist in removing the decal 22 from the slip paper 23 and attaching the decal 22 to the eye.

6. When the decal 22 has attached to the eye 26, the user can slowly peel the slip paper 23 away from the eye, beginning from the first end 28 of the decal 22 to the second end of the decal 22.

7. If the first end 28 of the decal 22 does not reach the inner corner of the eye 26, the user can fill in the inner corner of the eye 26 with an eyeliner after the decal 22 has been dried and set. In this way, the decal 22 can accommodate various eye shapes and sizes. Additionally, the user can apply other make up on top of the decal 22.

8. To remove the decal 22, the user can peel off or gently scratch off the decal 22 from the skin In this way, the present invention eliminates the need to use a make up remover or wash the decal 22 off of the skin.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An eyeliner composition, comprising:
20 to 60% by weight of composition glycerin;
0.1 to 10% by weight of composition colorant;
0.1 to 5% by weight of composition polypropylene;
0.1 to 5% by weight of composition triethanolamine;
20 to 60% by weight of composition latex; and
0.1 to 1% by weight composition polyether-modified polydimethylsiloxane.

2. The composition of claim 1, further comprising an adhesive agent, wherein the adhesive agent is present in an amount sufficient to allow the composition to adhere to skin.

3. The composition of claim 1, wherein said colorant is selected from the group consisting of carbon black, iron oxides, charcoals, micas, pearlescent pigments, metallic pigments, talc, clay, kaolin, zinc oxide, nylon powder, ultra marine, titanium dioxide, titanated mica, calcium carbonate, tar pigments, organic pigments, FD&C dyes, and D&C dyes.

4. The composition of claim 1, further comprising at least one nacreous pigment, wherein said at least one nacreous pigment is selected from the group consisting of titanium dioxide, bismuth oxychloride, iron oxide, titanium mica with iron oxides, titanium mica with ferric blue, titanium mica with chromium oxide, and titanium mica with an organic pigment.

5. An eyeliner composition, comprising:
at least one humectant;
at least one colorant;
at least one matting agent;
at least one pH modifier;
at least one film forming agent; and
at least one rheology modifier.

6. The composition of claim 5, wherein said at least one humectant comprises 20 to 80% by weight of composition glycerin.

7. The composition of claim 5, wherein said at least one matting agent comprises 0.1 to 5% by weight of composition polypropylene.

8. The composition of claim 5, wherein said at least one colorant is selected from the group consisting of carbon black and iron oxide black, said at least one colorant comprising 0.1 to 5% by weight of composition.

9. The composition of claim 5, further comprising 1 to 10% by weight of composition adhesive.

10. The composition of claim 5, wherein said at least one rheology modifier comprises 0.1% to 5% by weight composition polyether-modified polydimethylsiloxane.

11. The composition of claim 5, wherein said at least one pH modifier comprises 0.1 to 5% by weight of composition triethanolamine.

12. The composition of claim 5, wherein said at least one film forming agent comprises 20 to 60% by weight of composition latex.

13. An eyeliner decal, comprising:
a slip paper having a film-like eyeliner decal printed thereon, wherein said eyeliner decal is configured to be positioned along a rim of a human eyelid so as to enhance the appearance of said eyelid;
said eyeliner decal comprising a composition of humectant, colorant, matting agent, pH modifier, film forming agent, and rheology modifier, wherein said composition can transfer to said eyelid when applied thereto; and
a backing layer removably attached thereto said slip paper such that said backing layer covers said decal.

14. The eyeliner decal of claim 1, wherein said humectant comprises 20 to 60% by weight of composition glycerin.

15. The eyeliner decal of claim 1, wherein said colorant comprises 0.1 to 10% by weight of composition carbon black.

16. The eyeliner decal of claim 1, wherein said matting agent comprises 0.1 to 5% by weight of composition polypropylene.

17. The eyeliner decal of claim 1, wherein said pH modifier comprises 0.1 to 5% by weight of composition triethanolamine.

18. The eyeliner decal of claim 1, wherein said film forming agent comprises 20 to 60% by weight of composition latex.

19. The eyeliner decal of claim 1, wherein said rheology modifier comprises 0.1 to 1% by weight composition polyether-modified polydimethylsiloxane.
